SEQUENCE LISTING (9 sequences)

SEQ ID NO: 1 - DNA, 18 bp, synthetic construct
  source 1..18: mol_type = other DNA
cctcaaagccgagatcgc

SEQ ID NO: 2 - DNA, 21 bp, synthetic construct
  source 1..21: mol_type = other DNA
ctacgctgcagtcctcgctca

SEQ ID NO: 3 - DNA, 20 bp, synthetic construct
  source 1..20: mol_type = other DNA
aagatggccatcggatcctc

SEQ ID NO: 4 - DNA, 24 bp, synthetic construct
  source 1..24: mol_type = other DNA
ggtgctcttgaccaaattgggata

SEQ ID NO: 5 - DNA, 19 bp, synthetic construct
  source 1..19: mol_type = other DNA
cacattggcacccgcaatc

SEQ ID NO: 6 - DNA, 20 bp, synthetic construct
  source 1..20: mol_type = other DNA
gaggaacgagaagaggcttg

SEQ ID NO: 7 - DNA, 17 bp, synthetic construct
  source 1..17: mol_type = other DNA
ttcacgctcaccgtgcc

SEQ ID NO: 8 - DNA, 24 bp, synthetic construct
  source 1..24: mol_type = other DNA
ccaattcgagcagctgaaactgcg

SEQ ID NO: 9 - DNA, 25 bp, synthetic construct
  source 1..25: mol_type = other DNA
acttcctcaaggaacaacattgcca

(19) United States
(12) Patent Application Publication
(10) Pub. No.: US 2025/0019754 A1
(43) Pub. Date: Jan. 16, 2025

(54) METHOD FOR DETECTING MULTIPLE TARGETS IN SAME SAMPLE AND METHOD FOR QUANTIFYING CONCENTRATION OF MULTIPLE TARGETS IN SAME SAMPLE

(71) Applicant: Biotaitan Molmed Co. Ltd., Taipei City (TW)

(72) Inventors: Ching-Wei TSAI, Hsinchu County (TW); Yih-Jyh SHANN, Hsinchu County (TW); Szu-Yu LIU, Hsinchu County (TW); Li-Chi CHANG, Taipei City (TW)

(21) Appl. No.: 18/769,390

(22) Filed: Jul. 11, 2024

Related U.S. Application Data
(60) Provisional application No. 63/513,352, filed on Jul. 13, 2023.

(30) Foreign Application Priority Data
Jul. 4, 2024 (TW) 113125177

(51) Int. Cl. C12Q 1/6851; C12Q 1/6818; C12Q 1/6876

(52) U.S. Cl. CPC C12Q 1/6851; C12Q 1/6818; C12Q 1/6876; C12Q 2600/16; C12Q 2600/166

(57) ABSTRACT

A method for detecting multiple targets in the same sample includes the following steps. A sample is provided, and the sample includes a nucleic acid of at least one target. A reaction solution is provided, which includes target primer pairs and target fluorescent probes. The target fluorescent probes include at least two fluorophores. After mixing the sample and the reaction solution, a real-time polymerase chain reaction is performed. A fluorescence signal value ratio of the at least two fluorophores is calculated to determine whether the at least one target is present in the sample.

### RELATED APPLICATIONS

This application claims priority to U.S. Provisional Application Ser. No. 63/513,352, filed Jul. 13, 2023, and Taiwan Application Serial Number 113125177, filed Jul. 4, 2024, which are herein incorporated by reference.

### SEQUENCE LISTING XML

A sequence listing XML submitted as an xml file via PATENT CENTER is incorporated herein by reference. The sequence listing XML file submitted via PATENT CENTER with the name “CP-6182-US_SEQ_LIST” was created on Jul. 10, 2024, which is 9,107 bytes in size.

### BACKGROUND

### Technical Field

The present disclosure relates to a method for detecting multiple targets. More particularly, the present disclosure relates to a method for detecting the presence of multiple targets in a sample through a single polymerase chain reaction step, and a method for quantifying the concentration of multiple targets in the same sample.

### Description of Related Art

DNA and RNA are extremely important genetic materials in life phenomena. In the past, their related researches were limited by the low amount of DNA and RNA and were difficult to conduct. The invention of the polymerase chain reaction (PCR) has overcome the aforementioned difficulty, allowing the originally extremely low amount of the genetic material to be amplified to amounts that can be analyzed. PCR is limited to qualitative analysis, for example used for sequencing and gene selection. However, research on quantitative analysis has made major breakthroughs with the rapid development of quantitative polymerase chain reaction (qPCR) or real-time polymerase chain reaction (real-time PCR, RT-PCR).

Three basic steps are required to perform PCR. The first step is to extract and purify nucleic acids. The second step is to amplify or copy target nucleic acids, while specifically binding fluorescent probes or chemical probes to the target nucleic acids. The final step is detection using optical detection system, electrical detection system or other detection system, in which common detection systems include qPCR detection system, microarray detection system, nucleic acid sequencing detection system, etc. The qPCR technology is the most commonly used molecular diagnosis in point-of-care testing (POCT), which mainly uses fluorescent probes or fluorescent dyes to detect reaction products in real time. The fluorophore bound to a specific probe is used to generate fluorescence during the PCR process, and then a fluorescence detection system is used to detect the amount of fluorescence released by the fluorophore in each cycle. Then a content of the amplified products of each cycle is calculated to achieve the purpose of real-time quantification. Subsequently, it can be applied to quantify the number of specific genes in the genome, quantify the expression of specific genes, or detect variations in a single nucleic acid.

With the vigorous development of fields such as scientific research, precision medicine, personalized medicine, preventive medicine, pathological diagnosis (e.g. infectious diseases or oncology), human and animal disease control, drug discovery and screening, environmental monitoring and food safety, there is an urgent need for a method to analyze a large number of targets in a sample.

### SUMMARY

According to one embodiment of the present disclosure is to provide a method for detecting multiple targets in the same sample, which includes providing a sample, providing a reaction solution, performing a reaction step and performing a determining step. The sample includes a nucleic acid of at least one target. The reaction solution includes a primer pair set and a fluorescent probe set, the primer pair set includes a plurality of target primer pairs, the fluorescent probe set includes a plurality of target fluorescent probes and at least two fluorophores, and each of the at least two fluorophores are different. Each of the plurality of target fluorescent probes includes a target oligonucleotide bound to at least one of the at least two fluorophores. A number of the at least two fluorophores is m, a detectable number of the at least one target is 2^{m}−1, a number of the plurality of target primer pairs is 2^{m}−1, and a number of the plurality of target fluorescent probes is 2^{m}−1. Each of the plurality of target primer pairs has sequence specificity with one of the at least one target, and the target oligonucleotide of each of the plurality of target fluorescent probes has sequence specificity with one of the at least one target. In the reaction step, the sample and the reaction solution are mixed and a real-time polymerase chain reaction is performed to obtain a fluorescence signal value of the at least two fluorophores respectively. In the determining step, a fluorescence signal value ratio of the at least two fluorophores is calculated to determine whether the at least one target is present in the sample.

According to another embodiment of the present disclosure is to provide a method for quantifying concentration of multiple targets in the same sample, which includes performing a qualitative step and performing a quantitative step. In the qualitative step, whether the first target, the second target and/or the third target are present in the sample is determined by the method for detecting multiple targets in the same sample according to the foregoing embodiment. In the quantitative step, the first fluorescence signal value and the second fluorescence signal value are substituted into a calibration curve equation to calculate a concentration of the at least one target in the sample. The calibration curve equation is derived from a calibration curve set, and the calibration curve set includes a calibration curve for detecting the first target using the first fluorophore, a calibration curve for detecting the first target using the second fluorophore, a calibration curve for detecting the second target using the first fluorophore, a calibration curve for detecting the second target using the second fluorophore, a calibration curve for detecting the third target using the first fluorophore, and a calibration curve for detecting the third target using the second fluorophore.

### BRIEF DESCRIPTION OF THE DRAWINGS

The present disclosure can be more fully understood by reading the following detailed description of the embodiment, with reference made to the accompanying drawings as follows:

FIG. 1 is a step flow chart of a method for detecting multiple targets in the same sample according to one embodiment of the present disclosure.

FIG. 2 is a step flow chart of a method for quantifying concentration of multiple targets in the same sample according to another embodiment of the present disclosure.

FIG. 3A and FIG. 3B show a schematic diagram of predicted results and a diagram of actual experimental results of detection using the method for detecting multiple targets in the same sample of the present disclosure respectively.

FIG. 4A, FIG. 4B, FIG. 4C and FIG. 4D show schematic diagrams of predicted results of different samples when using two fluorophores for detection.

FIG. 5A, FIG. 5B, FIG. 5C and FIG. 5D show diagrams of actual experimental results of different samples when using two fluorophores for detection.

FIG. 6 shows a schematic diagram of predicted results of different samples when using four fluorophores for detection.

FIG. 7 shows calibration curve set results when using two fluorophores for detection.

FIG. 8A is a diagram showing the Ct value results of different samples after detection using the method for quantifying concentration of multiple targets in the same sample of the present disclosure.

FIG. 8B is a diagram showing converted results of sample copies of at least one target in different samples.

FIG. 9A, FIG. 9B and FIG. 9C show calibration curves of the third target and the first target respectively.

### DETAILED DESCRIPTION

Unless defined otherwise, all scientific or technical terms used herein have the same meaning as those understood by persons of ordinary skill in the art to which the present disclosure belongs. Any method and material similar or equivalent to those described herein can be understood and used by those of ordinary skill in the art to practice the present disclosure.

### A Method for Detecting Multiple Targets in the Same Sample

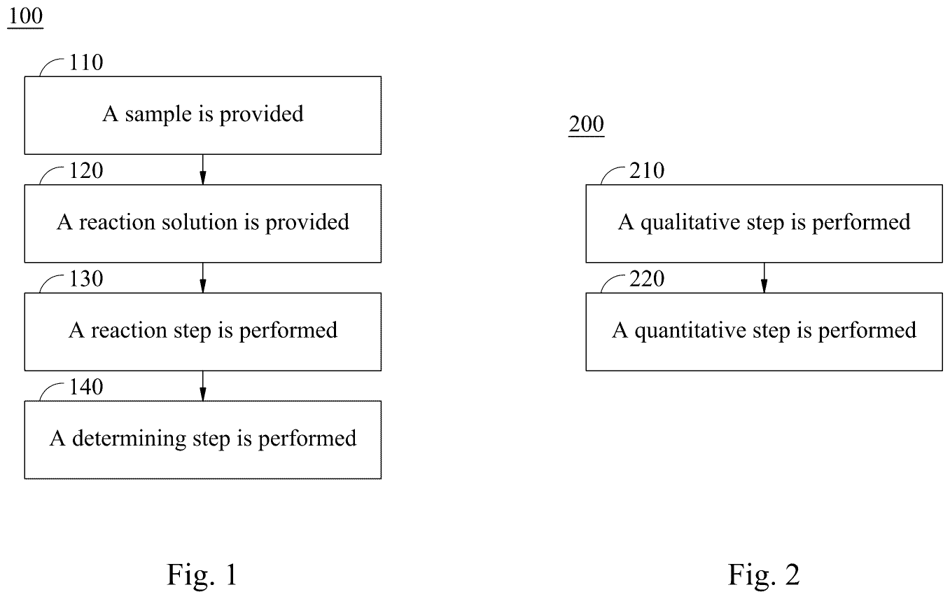

Reference is made to FIG. 1, which is a step flow chart of a method for detecting multiple targets in the same sample 100 according to one embodiment of the present disclosure. The method for detecting multiple targets in the same sample 100 includes Step 110, Step 120, Step 130 and Step 140.

In Step 110, a sample is provided. The sample includes a nucleic acid of at least one target.

In Step 120, a reaction solution is provided. The reaction solution includes a primer pair set and a fluorescent probe set, the primer pair set includes a plurality of target primer pairs, the fluorescent probe set includes a plurality of target fluorescent probes and at least two fluorophores, and each of the at least two fluorophores are different. Each of the plurality of target fluorescent probes includes a target oligonucleotide bound to at least one of the at least two fluorophores. A number of the at least two fluorophores is m, a detectable number of the at least one target is 2^{m}−1, a number of the plurality of target primer pairs is 2^{m}−1, and a number of the plurality of target fluorescent probes is 2^{m}−1. Each of the plurality of target primer pairs has sequence specificity with one of the at least one target, and the target oligonucleotide of each of the plurality of target fluorescent probes has sequence specificity with one of the at least one target.

For example, when the optical detection system has 2 fluorescent channels and the number of at least two fluorophores that can be used is 2, there are 4 combinations of different fluorophores. After deducting the internal control, the detectable number of the at least one target is 3. When the optical detection system has 3 fluorescent channels and the number of at least two fluorophores that can be used is 3, there are 8 combinations of different fluorophores. After deducting the internal control, the detectable number of the at least one target is 7. When the optical detection system has 4 fluorescent channels and the number of at least two fluorophores that can be used is 4, there are 16 combinations of different fluorophores. After deducting the internal control, the detectable number of the at least one target is 15. By analogy, it will not be repeated here.

When the number of the at least two fluorophores is 2, the at least one target can include a first target, a second target and a third target, the target primer pairs can include a first primer pair, a second primer pair and a third primer pair, the target fluorescent probes can include a first fluorescent probe, a second fluorescent probe and a third fluorescent probe, the at least two fluorophores can include a first fluorophore and a second fluorophore, and the first fluorophore and the second fluorophore are different. The first fluorescent probe includes a first oligonucleotide bound to the first fluorophore, the second fluorescent probe includes a second oligonucleotide bound to the second fluorophore, and the third fluorescent probe includes a third oligonucleotide bound to the first fluorophore and the third oligonucleotide bound to the second fluorophore. The first primer pair and the first oligonucleotide respectively have sequence specificity with the first target, the second primer pair and the second oligonucleotide respectively have sequence specificity with the second target, and the third primer pair and the third oligonucleotide respectively have sequence specificity with the third target.

In detail, a ratio of a content of the first fluorophore in the first fluorescent probe, a content of the second fluorophore in the second fluorescent probe and a total content of the first fluorophore and the second fluorophore in the third fluorescent probe can be 1:1:1, and a ratio of the first fluorophore to the second fluorophore in the third fluorescent probe can be 0.1:0.9 to 0.9:0.1. Furthermore, the first fluorescent probe, the second fluorescent probe and the third fluorescent probe respectively can include a fluorescent quenching group, and the at least two fluorophores and the fluorescent quenching group can be respectively bound to a 5′ end and a 3′ end of the first fluorescent probe, the second fluorescent probe and the third fluorescent probe. The at least two fluorophores can include at least two selected from a group consisting of FAM, FITC, Alexa 488, CF488, Atto 488, STAR 488, JOE, TET, Yakima Yellow, HEX, VIC, Atto 532, Alexa 532, Quasar 570, Cy3, TAMRA, ROX, Texas Red, Atto 590, Atto 594, Cy5, Atto 647, Alexa 647, Quasar 670 and Quasar 705. The fluorescent quenching group can be selected from a group consisting of BHQ-1, BHQ-2 and TAMRA. Reference is made to Table 1, which shows the combination of the fluorophores and the fluorescent quenching group in the target fluorescent probes.

TABLE 1

| | Maximum | Maximum | Fluorescence |
|---|---|---|---|
| | excitation | emission | quenching |
| Fluorophore | wavelength | wavelength | group |
| FAM, FITC, Alexa 488, | 494 nm | 520 nm | BHQ-1/TAMRA |
| CF488, Atto 488, | | | |
| STAR 488 | | | |
| JOE | 520 nm | 548 nm | BHQ-1/TAMRA |
| TET | 521 nm | 536 nm | BHQ-1/TAMRA |
| Yakima Yellow | 526 nm | 548 nm | BHQ-1/TAMRA |
| HEX, VIC, Atto 532, | 535 nm | 556 nm | BHQ-1/TAMRA |
| Alexa 532 | | | |
| Quasar 570 | 548 nm | 566 nm | BHQ-2 |
| Cy3 | 550 nm | 570 nm | BHQ-2 |
| TAMRA | 555 nm | 576 nm | BHQ-2 |
| ROX | 573 nm | 602 nm | BHQ-2 |
| Texas Red, Atto 590, | 583 nm | 603 nm | BHQ-2 |
| Atto 594 | | | |
| Cy5, Atto 647 | 651 nm | 674 nm | BHQ-2 |

In addition, the sample can include an internal control target as an internal control, the primer pair set can include an internal control primer pair, and the internal control primer pair has sequence specificity with the internal control target. The fluorescent probe set can include an internal control fluorescent probe, the internal control fluorescent probe includes an internal control oligonucleotide bound to the at least two fluorophores, and the internal control oligonucleotide has sequence specificity with the internal control target. Tx represents the at least one target, ic represents the internal control, Fm represents one of the at least two fluorophores, a content of each of the at least two fluorophores in the internal control fluorescent probe is α_{Fm}^{ic}, a content of each of the at least two fluorophores in each of the target fluorescent probes is β_{Fm}^{Tx}, and α_{Fm}^{Tx}:β_{Fm}^{Tx} can be 1:9, 2:8, 3:7, 4:6 or 5:5.

In Step 130, a reaction step is performed, in which the sample and the reaction solution are mixed and a real-time polymerase chain reaction is performed to obtain a fluorescence signal value of the at least two fluorophores respectively. When the number of the at least two fluorophores is 2, the fluorescence signal values of the at least two fluorophores include a first fluorescence signal value and a second fluorescence signal value.

In Step 140, a determining step is performed, in which a fluorescence signal value ratio of the at least two fluorophores is calculated to determine whether the at least one target is present in the sample. Furthermore, when the number of the at least two fluorophores is 2, the fluorescence signal values of the at least two fluorophores include a first fluorescence signal value and a second fluorescence signal value. If only the first fluorescent signal value is detected in the optical system, it means that only the first target is present in the sample. If only the second fluorescent signal value is detected, it means that only the second target is present in the sample. If a weak first fluorescent signal value and a weak second fluorescent signal value are detected simultaneously, it means that only the third target is present in the sample. If an additive first fluorescent signal value and the weak second fluorescent signal value are detected simultaneously, it means that the first target and the third target are present in the sample simultaneously. If the weak first fluorescent signal value and an additive second fluorescent signal value are detected simultaneously, it means that the second target and the third target are present in the sample simultaneously. If a strong first fluorescent signal value and a strong second fluorescent signal value are detected simultaneously, it means that the first target and the second target are present in the sample simultaneously. If the additive first fluorescent signal value and the additive second fluorescent signal value are detected simultaneously, it means that the first target, the second target and the third target are present in the sample simultaneously.

In detail, a ratio of a content of the first fluorophore in the first fluorescent probe, a content of the second fluorophore in the second fluorescent probe and a total content of the first fluorophore and the second fluorophore in the third fluorescent probe can be 1:1:1, and a ratio of the first fluorophore to the second fluorophore in the third fluorescent probe can be 0.1:0.9 to 0.9:0.1. When the fluorescence signal value ratio is 1:0, only the first target is present in the sample. When the fluorescence signal value ratio is 0:1, only the second target is present in the sample. When the fluorescence signal value ratio is 0.1:0.9 to 0.9:0.1, only the third target is present in the sample. When the fluorescence signal value ratio is 1.1:0.9 to 1.9:0.1, the first target and the third target are present in the sample. When the fluorescence signal value ratio is 1:1, the first target and the second target are present in the sample. When the fluorescence signal value ratio is 0.1:1.9 to 0.9:1.1, the second target and the third target are present in the sample. When the fluorescence signal value ratio is 1.1:1.9 to 1.9:1.1, the first target, the second target and the third target are present in the sample.

### A Method for Quantifying Concentration of Multiple Targets in the Same Sample

Reference is made to FIG. 2, which is a step flow chart of a method for quantifying concentration of multiple targets in the same sample 200 according to another embodiment of the present disclosure. The method for quantifying concentration of multiple targets in the same sample 200 includes Step 210 and Step 220.

In Step 210, a qualitative step is performed, in which whether the first target, the second target and/or the third target are present in the sample is determined by the method for detecting multiple targets in the same sample 100.

In Step 220, a quantitative step is performed, in which the first fluorescence signal value and the second fluorescence signal value are substituted into a calibration curve equation to calculate a concentration of the at least one target in the sample. The calibration curve equation is derived from a calibration curve set, and the calibration curve set includes a calibration curve for detecting the first target using the first fluorophore, a calibration curve for detecting the first target using the second fluorophore, a calibration curve for detecting the second target using the first fluorophore, a calibration curve for detecting the second target using the second fluorophore, a calibration curve for detecting the third target using the first fluorophore, and a calibration curve for detecting the third target using the second fluorophore.

In detail, when in the qualitative step, it is determined that the at least one target present in the sample is the first target, the second target or the third target, F1 represents the first fluorophore, F2 represents the second fluorophore, Tx represents the at least one target, and the calibration curve equation can be expressed as equation (1):

$${S}_{F 1}^{Tx} + {S}_{F 2}^{Tx} = {ε}_{F 1}^{Tx} [ Tx ] + {ε}_{F 2}^{Tx} [ Tx ] + {C}_{F 1}^{Tx} + {C}_{F 2}^{Tx} ; \qquad ( 1 )$$

where S_{F1}^{Tx} and S_{F2}^{Tx} are respectively the first fluorescence signal value and the second fluorescence signal value of the at least one target, [Tx] is the concentration of the at least one target, ϵ_{F1}^{Tx} and ϵ_{F2}^{Tx} are respectively a slope of a calibration curve for detecting the at least one target using the first fluorophore and a slope of a calibration curve for detecting the at least one target using the second fluorophore, and C_{F1}^{Tx} and C_{F2}^{Tx} are respectively an intercept of the calibration curve for detecting the at least one target using the first fluorophore and an intercept of the calibration curve for detecting the at least one target the second fluorophore.

When in the qualitative step, it is determined that the at least one target present in the sample are two of the first target, the second target and the third target, F1 represents the first fluorophore, F2 represents the second fluorophore, Tx represents one of the at least one target, Ty represents the other one of the at least one target, and the calibration curve equation can be expressed as equation (2) and equation (3):

$${S}_{F 1}^{Tx} + {S}_{F 1}^{Ty} = {ε}_{F 1}^{Tx} [ Tx ] + {ε}_{F 1}^{Ty} [ Ty ] + {C}_{F 1}^{Tx} + {C}_{F 1}^{Tx} ; \qquad ( 2 )$$

$${S}_{F 2}^{Tx} + {S}_{F 2}^{Ty} = {ε}_{F 2}^{Tx} [ Tx ] + {ε}_{F 2}^{Ty} [ Ty ] + {C}_{F 2}^{Tx} + {C}_{F 2}^{Tx} ; \qquad ( 3 )$$

where S_{F1}^{Tx}, S_{F1}^{Ty}, S_{F2}^{Tx} and S_{F2}^{Ty} are respectively a first fluorescence signal value of the one of the at least one target, a first fluorescence signal value of the other one of the at least one target, a second fluorescence signal value of the one of the at least one target, and a second fluorescence signal value of the other one of the at least one target, [Tx] and [Ty] are respectively a concentration of the one of the at least one target and a concentration of the other one of the at least one target, ϵ_{F1}^{Tx}, ϵ_{F1}^{Ty}, ϵ_{F2}^{Tx} and, ϵ_{F2}^{Ty} are respectively a slope of a calibration curve for detecting the one of at least one target using the first fluorophore, a slope of a calibration curve for detecting the other one of at least one target using the first fluorophore, a slope of a calibration curve for detecting the one of at least one target using the second fluorophore, and a slope of a calibration curve for detecting the other one of at least one target using the second fluorophore, and C_{F1}^{Tx}, C_{F1}^{Ty}, C_{F2}^{Tx} and C_{F2}^{Ty} are respectively an intercept of the calibration curve for detecting the one of at least one target using the first fluorophore, an intercept of the calibration curve for detecting the other one of at least one target using the first fluorophore, an intercept of the calibration curve for detecting the one of at least one target using the second fluorophore, and an intercept of the calibration curve for detecting the other one of at least one target using the second fluorophore.

When in the qualitative step, it is determined that the first target, the second target and the third target are present in the sample simultaneously, an auxiliary step can be further performed before the quantitative step. In the auxiliary step, the target fluorescent probes in the reaction solution are adjusted such that the first fluorescent probe includes the first oligonucleotide bound to the first fluorophore and the first oligonucleotide bound to the second fluorophore, or the second fluorescent probe includes the second oligonucleotide bound to the first fluorophore and the second oligonucleotide bound to the second fluorophore, and the third fluorescent probe includes the third oligonucleotide bound to the first fluorophore or the third oligonucleotide bound to the second fluorophore. Then the reaction solution and the sample are mixed, and the real-time polymerase chain reaction is performed to obtain the other first fluorescent signal value of the first fluorophore and the other second fluorescent signal value of the second fluorophore.

Furthermore, in the quantitative step, the first fluorescent signal value, the second fluorescent signal value, the other first fluorescent signal value and the other second fluorescent signal value obtained in the qualitative step and the auxiliary step are substituted into the calibration curve equation to calculate a concentration of the first target, a concentration of the second target and a concentration of the third target in the sample. F1 represents the first fluorophore, F2 represents the second fluorophore, T1 represents the first target, T2 represents the second target, T3 represents the third target, and the calibration curve equation can be expressed as equation (4) and equation (5):

$${S}_{F 1}^{T 1} + {S}_{F 1}^{T 2} + {S}_{F 1}^{T 3} = {ε}_{F 1}^{T 1} [ T 1 ] + {ε}_{F 1}^{T 2} [ T 2 ] + {ε}_{F 1}^{T 3} [ T 3 ] + {C}_{F 1}^{T 1} + {C}_{F 1}^{T 2} + {C}_{F 1}^{T 3} ; \qquad ( 4 )$$

$${S}_{F 2}^{T 1} + {S}_{F 2}^{T 2} + {S}_{F 2}^{T 3} = {ε}_{F 2}^{T 1} [ T 1 ] + {ε}_{F 2}^{T 2} [ T 2 ] + {ε}_{F 2}^{T 3} [ T 3 ] + {C}_{F 2}^{T 1} + {C}_{F 2}^{T 2} + {C}_{F 2}^{T 3} ; \qquad ( 5 )$$

where S_{F1}^{T1}, S_{F1}^{T2}, S_{F1}^{T3}, S_{F2}^{T1}, S_{F2}^{T2} and S_{F2}^{T3} are respectively the first fluorescence signal value of the first target, the first fluorescence signal value of the second target, the first fluorescence signal value of the third target, the second fluorescence signal value of the first target, the second fluorescence signal value of the second target, and the second fluorescence signal value of the third target, [T1], [T2] and [T3] are respectively the concentration of the first target, the concentration of the second target, and the concentration of the third target, ϵ_{F1}^{T1}, ϵ_{F1}^{T2}, ϵ_{F1}^{T3}, ϵ_{F2}^{T1}, ϵ_{F2}^{T2} and ϵ_{F2}^{T3} are respectively a slope of the calibration curve for detecting the first target using the first fluorophore, a slope of the calibration curve for detecting the second target using the first fluorophore, a slope of the calibration curve for detecting the third target using the first fluorophore, a slope of the calibration curve for detecting the first target using the second fluorophore, a slope of the calibration curve for detecting the second target using the second fluorophore, and a slope of the calibration curve for detecting the third target using the second fluorophore, and C_{F1}^{T1}, C_{F1}^{T2}, C_{F1}^{T3}, C_{F2}^{T1}, C_{F2}^{T2} and C_{F2}^{T3} are respectively an intercept of the calibration curve for detecting the first target using the first fluorophore, an intercept of the calibration curve for detecting the second target using the first fluorophore, an intercept of the calibration curve for detecting the third target using the first fluorophore, an intercept of the calibration curve for detecting the first target using the second fluorophore, an intercept of the calibration curve for detecting the second target using the second fluorophore, and an intercept of the calibration curve for detecting the third target using the second fluorophore.

### Test Example

### I. The Method for Detecting Multiple Targets in the Same Sample

In the experiment, influenza B virus was used as the first target (hereinafter referred to as T1), severe acute respiratory syndrome coronavirus 2 (SARS-CoV-2) was used as the second target (hereinafter referred to as T2) and type A Influenza virus was used as the third target (hereinafter referred to as T3). The first primer pair, the second primer pair and the third primer pair shown in Table 2 were used as the primer pair set, and the first fluorescent probe, the second fluorescent probe and the third fluorescent probe shown in Table 3 were used as the fluorescent probe set. After adding reaction enzymes and reaction reagents, the real-time polymerase chain reaction was performed. The reaction conditions were denaturation at 95° C. for 1 minute, followed by 44 thermal cycles to analyze whether T1, T2 and/or T3 were present in the sample. Each thermal cycle includes denaturation at 95° C. for 10 seconds and adhesion at 65° C. for 30 seconds. In addition, a group without target template was used as the internal control (ic).

TABLE 2

| | Forward primer | Reverse primer |
|---|---|---|
| First primer pair | SEQ ID NO: 1 | SEQ ID NO: 2 |
| Second primer pair | SEQ ID NO: 3 | SEQ ID NO: 4 |
| Third primer pair | SEQ ID NO: 5 | SEQ ID NO: 6 |

TABLE 3

| | Target | First | Second |
|---|---|---|---|
| | oligonucleotide | fluorophore | fluorophore |
| First fluorescent probe | SEQ ID NO: 7 | HEX | x |
| Second fluorescent probe | SEQ ID NO: 8 | x | FAM |
| Third fluorescent probe | SEQ ID NO: 9 | 0.5 × HEX | 0.5 × FAM |

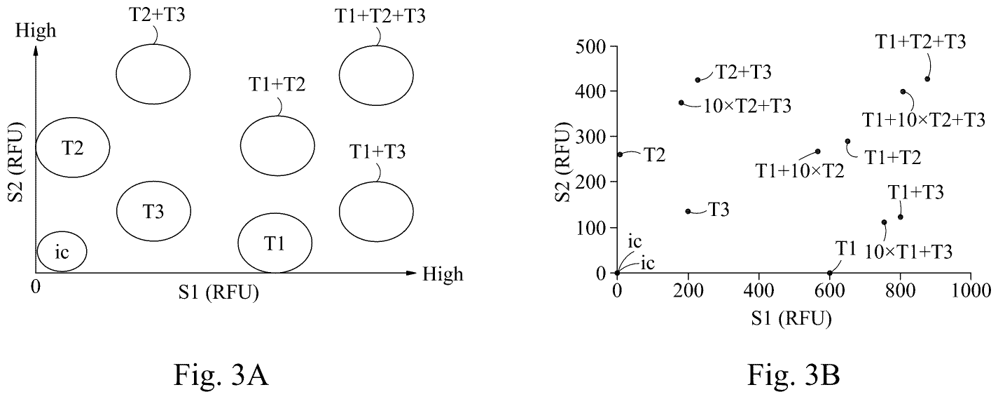

Reference is made to FIG. 3A and FIG. 3B, which show a schematic diagram of predicted results and a diagram of actual experimental results of detection using the method for detecting multiple targets in the same sample of the present disclosure respectively. As shown in the schematic diagram of the prediction results in FIG. 3A, using the first fluorophore and the second fluorophore (hereinafter referred to as F1 and F2) for detection, the first fluorescent signal value and the second fluorescent signal value (hereinafter referred to as S1 and S2) can be obtained. Then, the different targets present in the sample are determined by calculating the fluorescence signal value ratio of S1 and S2. For example, in FIG. 3A, F1:F2 labeled on T3 is 0.5:0.5, F1:F2 labeled on T1 is 1:0, and F1:F2 labeled on T2 is 0:1. If only T3 is present in the sample, the detected S1:S2 is 0.5:0.5. If only T1 is present in the sample, the detected S1:S2 is 1:0. If only T2 is present in the sample, the detected S1:S2 is 0:1. If T1 and T3 (T1+T3) are present in the sample simultaneously, the additive S1 and the weak S2 can be detected, that is, S1:S2 is 1.5:0.5. If T2 and T3 (T2+T3) are present in the sample simultaneously, the weak S1 and the additive S2 can be detected, that is, S1:S2 is 0.5:1.5. If T1 and T2 (T1+T2) are present in the sample simultaneously, S1 and S2 of the same intensity can be detected, that is, S1:S2 is 1:1. If T1, T2 and T3 (T1+T2+T3) are present in the sample simultaneously, the additive S1 and the additive S2 can be detected, that is, S1:S2 is 1.5:1.5.

As shown in diagram of actual experimental results in FIG. 3B, the actual experimental results are consistent with the predicted results. When two or more targets are present in the sample simultaneously, even if the concentration of one of the targets increases 10 times, the detected fluorescence signal value will not increase accordingly. For example, when the sample includes T1 with 10-fold concentration and T3 (10×T1+T3), the additive S1 and the weak S2 can be detected, that is, S1:S2 is 1.5:0.5. The same phenomenon can also be observed in other groups, indicating that S1 and S2 will not be interfered by the different concentrations of the target in the sample, and the target present in the sample can be correctly determined.

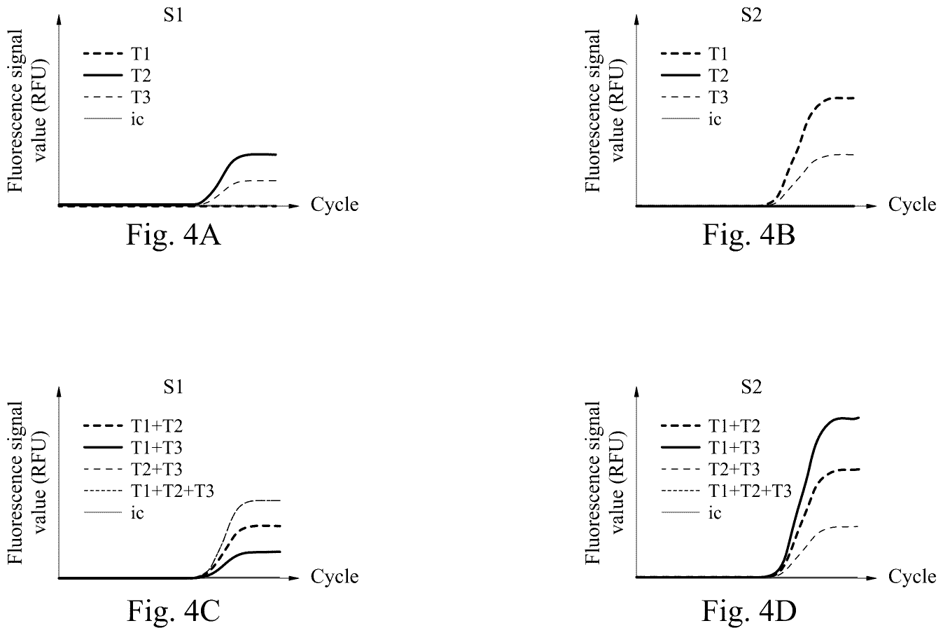

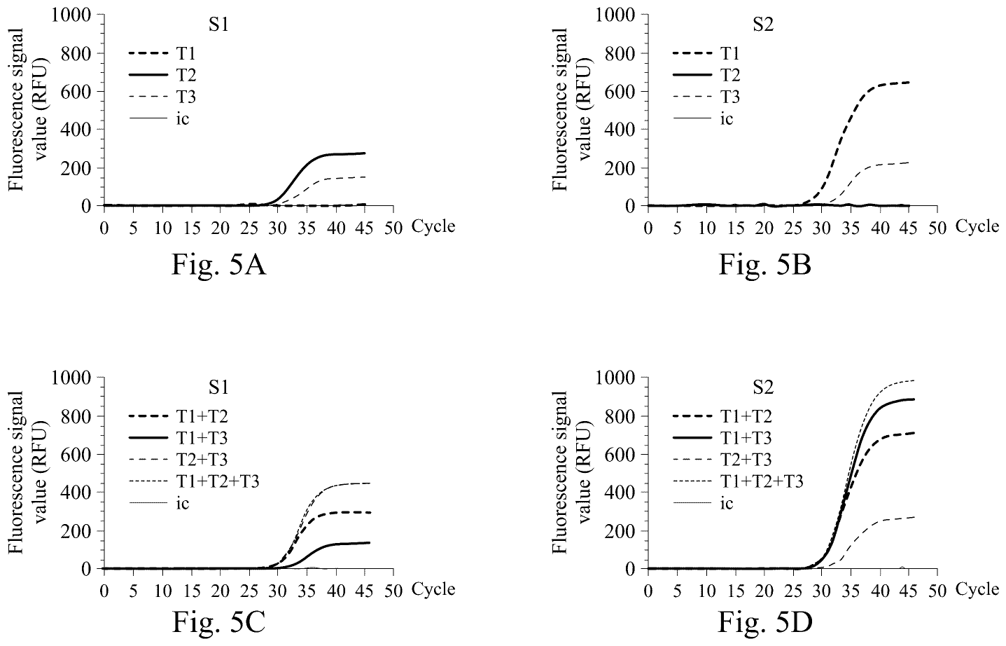

Reference is made to FIG. 4A to FIG. 5D. FIG. 4A, FIG. 4B, FIG. 4C and FIG. 4D show schematic diagrams of predicted results of different samples when using two fluorophores for detection. FIG. 5A, FIG. 5B, FIG. 5C and FIG. 5D show diagrams of actual experimental results of different samples when using two fluorophores for detection. In FIG. 4A, FIG. 4B, FIG. 5A and FIG. 5B, only one target, T1, T2 or T3, is present in the sample. FIG. 4A and FIG. 5A show the prediction results and detection results of S1 respectively, and FIG. 4B and FIG. 5B show the prediction results and detection results of S2 respectively. In FIG. 4C, FIG. 4D, FIG. 5C and FIG. 5D, two or more target are present in the sample, including T1+T2, T1+T3, T2+T3 or T1+T2+T3. FIG. 4C and FIG. 5C show the prediction results and detection results of S1 respectively, and FIG. 4D and FIG. 5D show the prediction results and detection results of S2 respectively. As shown in FIG. 4A to FIG. 5D, the schematic diagram of the prediction results and the diagram of the actual experimental results have the same trends and results in each sample, proving the feasibility and accuracy of the method for detecting multiple targets in the same sample of the present disclosure. Different fluorophores can be used to label the targets in the sample, different targets in the sample can be distinguished by matching different fluorophores with different proportions, and whether multiple targets are present in the sample simultaneously can be detected.

In addition, when the optical detection system can simultaneously detect the fluorescence signals of three different fluorophores, three fluorophores (hereinafter referred to as F1, F2 and F3) can be used for detection, and up to 7 targets can be labeled (the first target to the seventh target are referred to as T1 to T7). The targets are labelled with different combinations of fluorophores and then analyzed and measured. For example, T1 is labeled with F1, T2 is labeled with F2, T3 is labeled with F3, T4 is labeled with F1 and F2, T5 is labeled with F1 and F3, T6 is labeled with F2 and F3, and T7 is labeled with F1, F2, and F3. Ic represents the internal control, and the fluorescence signal values of the internal control in the three fluorescence channels are α_{F1}^{ic}S_{F1}^{ic}, α_{F2}^{ic}S_{F2}^{ic} and α_{F3}^{ic}S_{F3}^{ic} respectively. The fluorescence signal values of T1 to T7 are β_{F1}^{T1}S_{F1}^{T1}, β_{F2}^{T2}S_{F2}^{T2}, β_{F3}^{T3}S_{F3}^{T3}, β_{F1}^{T4}S_{F1}^{T4}+β_{F2}^{T4}S_{F2}^{T4}, β_{F1}^{T5}S_{F1}^{T5}+β_{F3}^{T5}S_{F3}^{T5}, β_{F2}^{T6}S_{F2}^{T6}+β_{F3}^{T6}S_{F3}^{T6} and β_{F1}^{T7}S_{F1}^{T7}+β_{F2}^{T7}S_{F2}^{T7}+β_{F3}^{T7}S_{F3}^{T7} respectively. If only T1 is present in the sample, the fluorescence signal values of F1 channel, F2 channel and F3 channel are α_{F1}^{ic}S_{F1}^{ic}+β_{F1}^{T1}S_{F1}^{T1}, α_{F2}^{ic}S_{F2}^{ic} and α_{F3}^{ic} S_{F3}^{ic}. If T1, T2 and T4 are present in the sample, the fluorescence signal values of F1 channel, F2 channel and F3 channel are 60 _{F1}^{ic} S_{F1}^{ic}+β_{F1}^{T1} S_{F1}^{T1}+β_{F1}^{T4} S_{F1}^{T4}, α_{F2}^{ic}S_{F2}^{ic}+β_{F2}^{T2}S_{F2}^{T2}+β_{F2}^{T4}S_{F2}^{T3} and α_{F2}^{ic}S_{F3}^{ic}. Furthermore, the ratio of two or more fluorophores labeled on T1 to T7 can be adjusted. For example, the ratio of F1 to F3 labeled on T6 can be 1:1, 1:2, 1:4 or 1:8.

When the optical detection system can simultaneously detect the fluorescence signals of four different fluorophores, four fluorophores (hereinafter referred to as F1, F2, F3 and F4) can be used for detection, and up to 15 targets can be labeled (the first target to the fifteenth target are referred to as T1 to T15). The targets are labelled with different combinations of fluorophores and then analyzed and measured. In the experiment, an internal control target with a known sequence can be used as the internal control, and the nucleic acid of the internal control target can be controlled at a copy number of 100-10,000. The ratio of the fluorophores in different target fluorescent probes can be adjusted to the concentrations where the fluorescence signal values of F1, F2, F3 and F4 can be detected, but the fluorescence signal value of the target is lower than that of the positive control at relatively high cycle values to confirm that the qPCR detection is working properly. It can be subsequently used to adjust the target probe, which can correspond to a single target fluorescent probe or two or more target fluorescent probes. Ic represents the internal control, and Fm represents one of the fluorophores (m=1−4). The content of each fluorophore in the internal control fluorescence probe is α_{Fm}^{ic}, that is, the signal values of the internal control in the four fluorescence channels are α_{F1}^{ic}S_{F1}^{ic}, α_{F2}^{ic}S_{F2}^{ic}, α_{F3}^{ic}S_{F3}^{ic} and α_{F4}^{ic}S_{F4}^{ic} respectively. Tx represents the at least one target (x=1−15), the content of each fluorophore in each target fluorescence probe is β_{Fm}^{Tx}, that is, the fluorescence signal values of the at least one target in F1 channel, F2 channel, F3 channel and F4 channel are β_{F1}^{Tx}S_{F1}^{Tx}, β_{F2}^{Tx}S_{F2}^{Tx}, β_{F3}^{Tx}S_{F3}^{Tx} and β_{F4}^{Tx}S_{F4}^{Tx} respectively, and α_{Fm}^{ic}:β_{Fm}^{Tx} can be 1:9, 2:8, 3:7, 4:6 or 5:5. When the same target has two or more fluorescence signal values, the proportion of the fluorophores in the internal control fluorescent probe can be adjusted so that the fluorescence signal values of the internal control are α_{F1}^{Tx}S_{F1}^{Tx}, α_{F2}^{Tx}S_{F2}^{Tx}, α_{F3}^{Tx}S_{F3}^{Tx} and α_{F4}^{Tx}S_{F4}^{Tx} respectively. At the same time, the proportion of the fluorophores in different targets can be adjusted so that the fluorescence signal values of the at least one target are β_{F1}^{Tx}S_{F1}^{Tx}, β_{F2}^{Tx}S_{F2}^{Tx}, β_{F3}^{Tx}S_{F3}^{Tx} and β_{F4}^{Tx}S_{F4}^{Tx} respectively. The fluorescence signal values that can be detected in each fluorescence channel from T1 to T15 are shown in Table 4.

TABLE 4

| | F1 | F2 | F3 | F4 |
|---|---|---|---|---|
| ic | α_{F1}^{ic} S_{F1}^{ic} | α_{F2}^{ic} S_{F2}^{ic} | α_{F3}^{ic} S_{F3}^{ic} | α_{F4}^{ic} S_{F4}^{ic} |
| T1 | β_{F1}^{T1} S_{F1}^{T1} | x | x | x |
| T2 | x | β_{F2}^{T2} S_{F2}^{T2} | x | x |
| T3 | x | x | β_{F3}^{T3} S_{F3}^{T3} | x |
| T4 | x | x | x | β_{F4}^{T4} S_{F4}^{T4} |
| T5 | β_{F1}^{T5} S_{F1}^{T5} | β_{F2}^{T5} S_{F2}^{T5} | x | x |
| T6 | β_{F1}^{T6} S_{F1}^{T6} | x | β_{F3}^{T6} S_{F3}^{T6} | x |
| T7 | β_{F1}^{T7} S_{F1}^{T7} | x | x | β_{F4}^{T7} S_{F4}^{T7} |
| T8 | x | β_{F2}^{T8} S_{F2}^{T8} | β_{F3}^{T8} S_{F3}^{T8} | x |
| T9 | x | β_{F2}^{T9} S_{F2}^{T9} | x | β_{F4}^{T9} S_{F4}^{T9} |
| T10 | x | x | β_{F3}^{T10} S_{F3}^{T10} | β_{F4}^{T10} S_{F4}^{T10} |
| T11 | x | β_{F2}^{T11} S_{F2}^{T11} | β_{F3}^{T11} S_{F3}^{T11} | β_{F4}^{T11} S_{F4}^{T11} |
| T12 | β_{F1}^{T12} S_{F1}^{T12} | x | β_{F3}^{T12} S_{F3}^{T12} | β_{F4}^{T12} S_{F4}^{T12} |
| T13 | β_{F1}^{T13} S_{F1}^{T13} | β_{F2}^{T13} S_{F2}^{T13} | x | β_{F4}^{T13} S_{F4}^{T13} |
| T14 | β_{F1}^{T14} S_{F1}^{T14} | β_{F2}^{T14} S_{F2}^{T14} | β_{F3}^{T14} S_{F3}^{T14} | x |
| T15 | β_{F1}^{T15} S_{F1}^{T15} | β_{F2}^{T15} S_{F2}^{T15} | β_{F3}^{T15} S_{F3}^{T15} | β_{F4}^{T15} S_{F4}^{T15} |

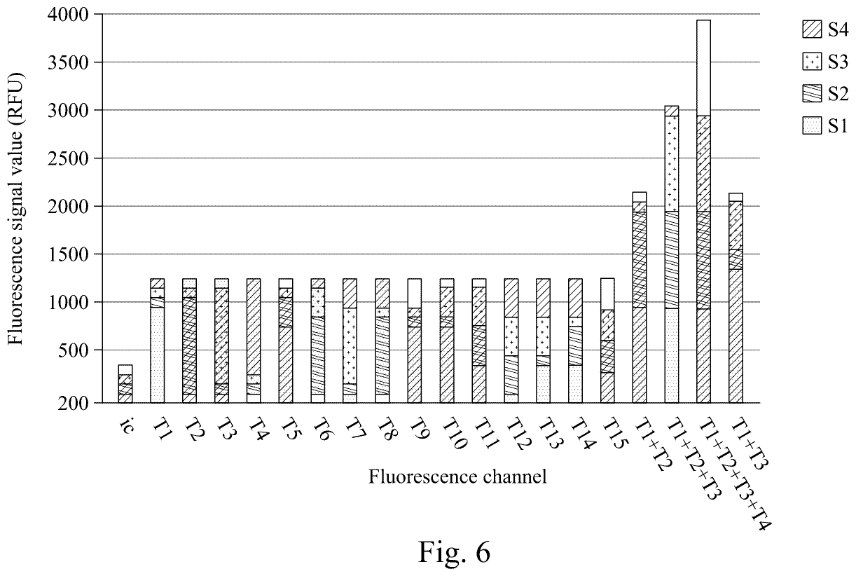

Reference is made to FIG. 6, which shows a schematic diagram of predicted results of different samples when using four fluorophores for detection. As shown in Table 4 and FIG. 6, if only T1 is present in the sample, the fluorescence signal values of F1 channel, F2 channel, F3 channel and F4 channel are α_{F1}^{ic}S_{F1}^{ic}+β_{F1}^{T1}S_{F1}^{T1}, α_{F2}^{ic}S_{F2}^{ic}, α_{F3}^{ic}S_{F3}^{ic} and α_{F4}^{ic}S_{F4}^{ic}. If T1 and T5 are present in the sample simultaneously, the fluorescence signal values of F1 channel, F2 channel, F3 channel and F4 channel are α_{F1}^{ic}S_{F1}^{ic}+β_{F1}^{T1}S_{F1}^{T1}+β_{F1}^{T5}S_{F1}^{T5}, α_{F2}^{ic}S_{F2}^{ic}+β_{F2}^{T5}S_{F2}^{T5}, α_{F3}^{ic}S_{F3}^{ic} and α_{F4}^{ic}S_{F4}^{ic}. Therefore, the detected fluorescence signal values in F1 channel, F2 channel, F3 channel and F4 channel can be used to determine whether the multiple targets are present in the sample.

### II. The Method for Quantifying Concentration of Multiple Targets in the Same Sample

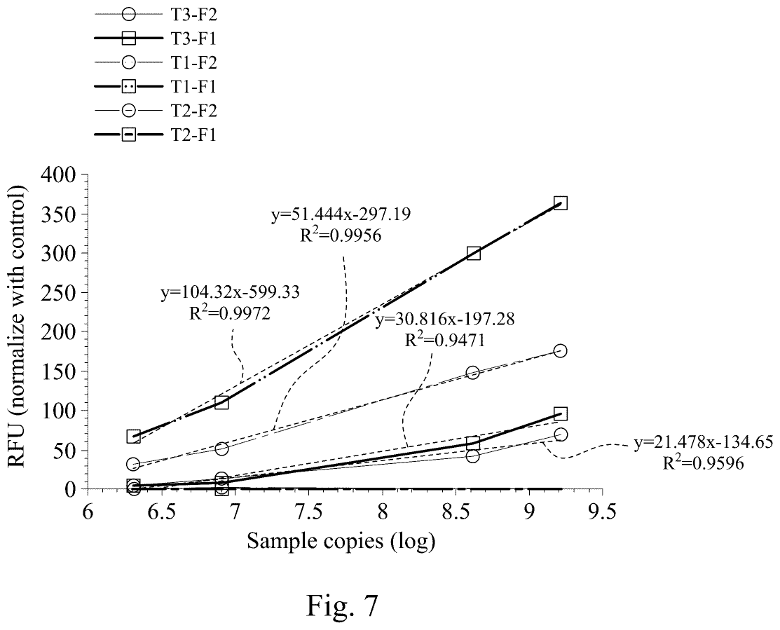

To verify feasibility and accuracy of the method of quantifying concentration of multiple targets in the same sample of the present disclosure, influenza B virus was used as the first target (hereinafter referred to as T1), severe acute respiratory syndrome coronavirus 2 (SARS-CoV-2) was used as the second target (hereinafter referred to as T2) and type A Influenza virus was used as the third target (hereinafter referred to as T3). The first primer pair, the second primer pair and the third primer pair shown in Table 2 were used as the primer pair set, and the first fluorescent probe, the second fluorescent probe and the third fluorescent probe shown in Table 3 were used as the fluorescent probe set. After adding reaction enzymes and reaction reagents, the real-time polymerase chain reaction was performed. The reaction conditions were denaturation at 95° C. for 1 minute, followed by 44 thermal cycles. Each thermal cycle includes denaturation at 95° C. for 10 seconds and adhesion at 65° C. for 30 seconds. In addition, a group without target template was used as the internal control (ic). In the experiment, T1, T2 and T3 with known concentrations were used as standards, and performed real-time polymerase chain reaction with the target fluorescent probe bound to the first fluorophore (hereinafter referred to as F1) and the target fluorescent probe bound to the second fluorophore (hereinafter referred to as F2) respectively. Then, the calibration curves made from the obtained reaction product detection curves are combined into a calibration curve set to confirm that T1, T2, and T3 can be accurately quantified. Reference is made to FIG. 7, which shows calibration curve set results when using two fluorophores for detection. HEX was used as F1 and FAM was used as F2 to execute the calibration curves of T1, T2 and T3. Because T1 was not labelled with F2 and T2 was not labelled with F1, there are only 4 calibration curves in FIG. 7.

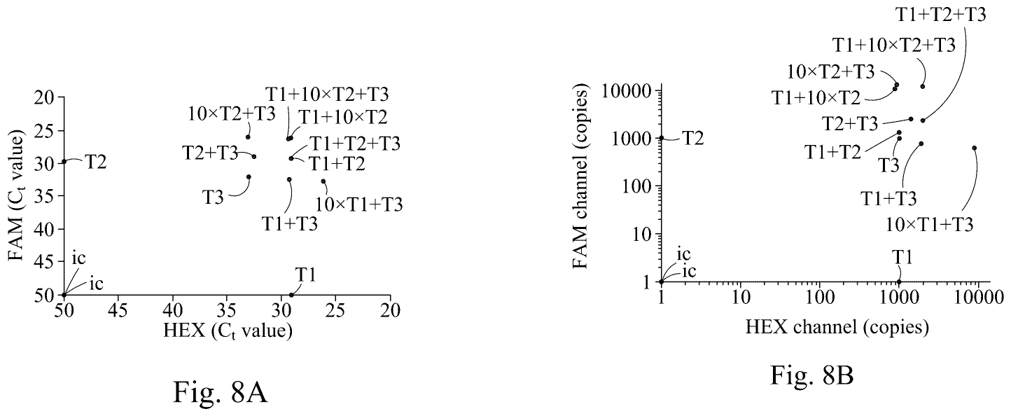

Reference is made to FIG. 8A and FIG. 8B, FIG. 8A is a diagram showing the Ct value results of different samples after detection using the method for quantifying concentration of multiple targets in the same sample of the present disclosure, and FIG. 8B is a diagram showing converted results of sample copies of at least one target in different samples. In FIG. 8A and FIG. 8B, F1:F2 labeled on T3 is 0.5:0.5, F1:F2 labeled on T1 is 1:0, and F1:F2 labeled on T2 is 0:1. As the concentration of at least one target increases, the corresponding fluorescence signal value (including the first fluorescence signal value and the second fluorescence signal value, hereinafter referred to as S1 and S2) also increases.

As shown in the Ct value results in FIG. 8A, if only T3 is present in the sample, the Ct values of S1 and S2 can be detected (the Ct value of S1 is 33 and the Ct value of S2 is 32). If only T1 is present in the sample, only the Ct value of S1 can be detected (the Ct value is 29.1). If only T2 is present in the sample, only the Ct value of S2 can be detected (the Ct value is 29.7). If T1 and T3 (T1+T3) are present in the sample simultaneously, because the Ct value of S1 of T3 appears later than the Ct value of S1 of T1, only the earlier Ct value will appear under the same fluorescent Ct value, the Ct value of S1 of T1 and the Ct value of S1 of T3 can be detected (the Ct value of S1 is 29.2 and the Ct value of S2 is 32.4). If T2 and T3 (T2+T3) are present in the sample simultaneously, the Ct value of S1 of T3 and the Ct value of S2 of T2 can be detected (the Ct value of S1 is 32.5 and the Ct value of S2 is 29). If T1 and T2 (T1+T2) are present in the sample simultaneously, the Ct value of S1 of T1 and the Ct value of S2 of T2 can be detected (the Ct value of S1 is 29.1 and the Ct value of S2 is 29.3).

Furthermore, multiple target tests at different concentrations were performed on the aforementioned basis. As shown in the results, if T3 and T1 with 10-fold concentration (10×T1+T3) are present in the sample simultaneously, the Ct value of S1 of T1 will appear early (the Ct value of S1 is 26.1 and the Ct value of S2 is 32.7). If T3 and T2 with 10-fold concentration (10×T2+T3) are present in the sample simultaneously, the Ct value of S2 of T2 will appear early (the Ct value of S1 is 33 and the Ct value of S2 is 26.1). If T1 and T2 with 10-fold concentration (T1+10×T2) are present in the sample simultaneously, the Ct value of S2 of T2 will appear early (the Ct value of S1 is 29.3 and the Ct value of S2 is 26.3). If T1, T2 and T3 are present in the sample simultaneously, the Ct value of S1 of T1 and the Ct value of S2 of T2 can be detected (the Ct value of S1 is 29.1 and the Ct value of S2 is 29.3). If T1, T2 with 10-fold concentration and T3 (T1+10×T2+T3) are present in the sample simultaneously, the detection results are the Ct value of S1 of T1 that is maintained and the Ct value of S2 of T2 that appears early (the Ct value of S1 is 29.1 and the Ct value of S2 is 26.2).

Then substituting the Ct values of S1 and S2 of the different samples obtained in FIG. 8A into the calibration curve set in FIG. 7 to convert the sample copies of at least one target in different samples. The results are shown in FIG. 8B.

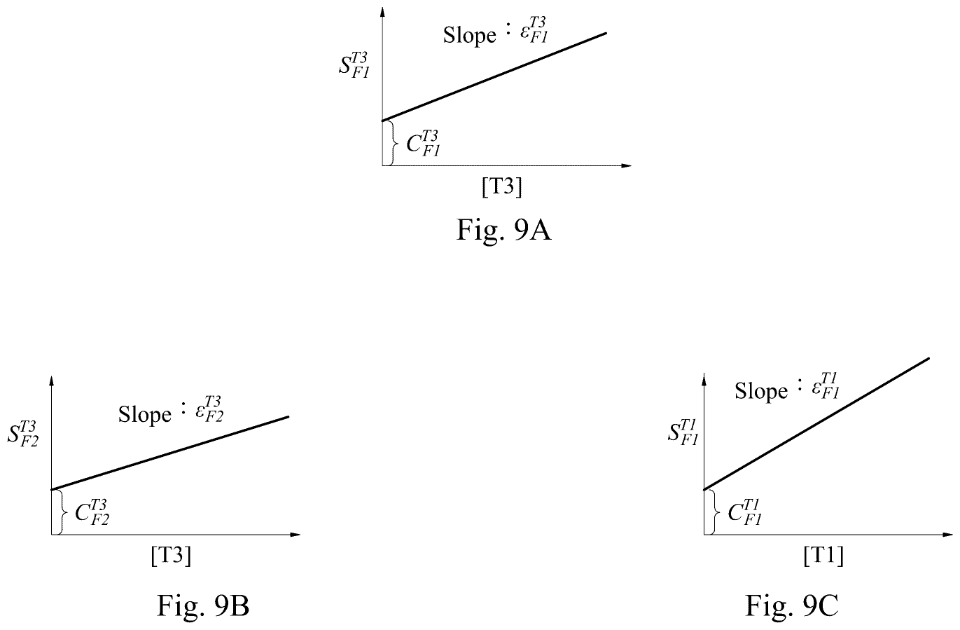

To further explain the qualitative step in the method for quantifying concentration of multiple targets in the same sample of the present disclosure, reference is made to FIG. 9A to FIG. 9C, which show calibration curves of the third target (T3) and the first target (T1) respectively. FIG. 9A and FIG. 9B are calibration curves for detecting T3 using the first fluorophore (F1) and the second fluorophore (F2) respectively. FIG. 9C is a calibration curve for detecting T1 using F1. Assume that F1:F2 labeled on T3 is 0.5:0.5 and F1:F2 labeled on T1 is 1:0. The results show that the first fluorescent signal value (S1) detected by the sample is 1.5, and the second fluorescent signal value (S2) is 0.5. Using the method for detecting multiple targets in the same sample of the present disclosure, it can be known that T1 and T3 are present in the sample simultaneously. Tx can be set to T1 or T3 and substituted into the aforementioned equation (1), as shown in the following equation (6) and equation (7):

$${S}_{F 1}^{T 1} + {S}_{F 2}^{T 1} = {ε}_{F 1}^{T 1} [ T 1 ] + {ε}_{F 2}^{T 1} [ T 1 ] + {C}_{F 1}^{T 1} + {C}_{F 2}^{T 1} ; \qquad ( 6 )$$

$${S}_{F 1}^{T 3} + {S}_{F 2}^{T 3} = {ε}_{F 1}^{T 3} [ T 3 ] + {ε}_{F 2}^{T 3} [ T 3 ] + {C}_{F 1}^{T 3} + {C}_{F 2}^{T 3} ; \qquad ( 7 )$$

then the detected S1 is substituted into S_{F1}^{T1} and S_{F1}^{T3}, and the detected S2 is substituted into S_{F2}^{T1} and S_{F2}^{T3}, ϵ_{F1}^{T3}[T3], is equal to ϵ_{F2}^{T3}[T3], so the concentration of T3 can be obtained. S1 of T1 is 0, so the concentration of T1 can be obtained.

That is, if it is known that T1 and T3 are present in the sample simultaneously, Tx can be set to T3 and Ty can be set to T1 and substituted into the aforementioned equation (2) and equation (3), as shown in the following equation (8) and equation (9):

$${S}_{F 1}^{T 3} + {S}_{F 1}^{T 1} = {ε}_{F 1}^{T 3} [ T 3 ] + {ε}_{F 1}^{T 1} [ T 1 ] + {C}_{F 1}^{T 3} + {C}_{F 1}^{T 1} ; \qquad ( 8 )$$

$${S}_{F 2}^{T 3} + {S}_{F 2}^{T 1} = {ε}_{F 2}^{T 3} [ T 3 ] + {ε}_{F 2}^{T 1} [ T 1 ] + {C}_{F 2}^{T 3} + {C}_{F 2}^{T 1} . \qquad ( 9 )$$

Then the detected S1 is substituted into and, and the detected S2 is SF1 T3 substituted into and to calculate the concentration of T1 and the concentration of T3.

Furthermore, the calibration curve equation in the quantitative step of the method for quantifying concentration of multiple targets in the same sample depends on the determined result in the qualitative step. If the determined result shows that only single target (T1, T2 or T3) is present in the sample, the detection results are substituted into the calibration curve equation shown in the aforementioned equation (1) to calculate the concentration of the at least one target. If the determined result shows that two targets (two of T1, T2 and T3) are present in the sample simultaneously, the detection results are substituted into the calibration curve equation shown in the aforementioned equation (2) and equation (3) to calculate the concentration of the two targets. If the determined result shows that T1, T2 and T3 are present in the sample simultaneously, the auxiliary step is performed first. For example, the first fluorescent probe, the second fluorescent probe and the third fluorescent probe are adjusted as shown in Table 5, other test conditions are the same as the qualitative step, and another real-time polymerase chain reaction is performed to obtain another first fluorescent signal value (S1′) and another second fluorescent signal value (S2′). S1, S1′, S2 and S2′ are substituted into the calibration curve equation shown in the aforementioned equation (4) and equation (5) to calculate the concentration of T1, the concentration of T2 and the concentration of T3.

TABLE 5

| | Target | First | Second |
|---|---|---|---|
| | oligonucleotide | fluorophore | fluorophore |
| First fluorescent probe | SEQ ID NO: 7 | HEX | x |
| Second fluorescent probe | SEQ ID NO: 8 | 0.5 × HEX | 0.5 × FAM |
| Third fluorescent probe | SEQ ID NO: 9 | x | FAM |

In summary, the method for detecting multiple targets in the same sample of the present disclosure can detect whether multiple targets are present in the same sample through a single polymerase chain reaction step by regulating the combination and proportion of the fluorophores in the reaction solution. For this purpose, a set of multiplex detection reagents can be established. In addition, the method for quantifying concentration of multiple targets in the same sample of the present disclosure uses the qualitative step to determine the multiple targets present in the sample, and then substitutes the detected first fluorescence signal value and the second fluorescence signal value into the calibration curve equation to quantify concentration of multiple targets in the same sample simultaneously, which can increase test output and reduce costs. Taking common respiratory viruses as an example, multiple viruses are prevalent at the same time every autumn and winter. If a patient has cold symptoms, in order to screen for viruses with corresponding drugs at the clinical end, multiple tests must be taken at the same time, and rapid test kit often has false negatives. The method for detecting multiple targets in the same sample and the method for quantifying concentration of multiple targets in the same sample of the present disclosure can simultaneously detect multiple viruses such as influenza virus, parainfluenza virus, respiratory fusion virus, adenovirus and coronavirus. Furthermore, the method for detecting multiple targets in the same sample and the method for quantifying concentration of multiple targets in the same sample of the present disclosure have the potential to be applied to platforms of immune diseases and cancer screening, such as detection of 14 types of high-risk HPV simultaneously or detection of single nucleotide polymorphisms, which is of great help to the clinical end and can be used as a very powerful molecular diagnostic platform.

Although the present disclosure has been described in considerable detail with reference to certain embodiments thereof, other embodiments are possible. Therefore, the spirit and scope of the appended claims should not be limited to the description of the embodiments contained herein. In view of the foregoing, it is intended that the present disclosure cover modifications and variations of this disclosure provided they fall within the scope of the following claims.

What is claimed is:

1. A method for detecting multiple targets in the same sample, comprising:
providing a sample, wherein the sample comprises a nucleic acid of at least one target;
providing a reaction solution, wherein the reaction solution comprises:
a primer pair set comprising a plurality of target primer pairs; and
a fluorescent probe set comprising a plurality of target fluorescent probes and at least two fluorophores, and each of the at least two fluorophores being different, wherein each of the plurality of target fluorescent probes comprises a target oligonucleotide bound to at least one of the at least two fluorophores;
wherein a number of the at least two fluorophores is m, a detectable number of the at least one target is 2^{m}−1, a number of the plurality of target primer pairs is 2^{m}−1, and a number of the plurality of target fluorescent probes is 2^{m}−1;
wherein each of the plurality of target primer pairs has sequence specificity with one of the at least one target, and the target oligonucleotide of each of the plurality of target fluorescent probes has sequence specificity with one of the at least one target;
performing a reaction step, wherein the sample and the reaction solution are mixed and a real-time polymerase chain reaction is performed to obtain fluorescence signal values of the at least two fluorophores respectively; and
performing a determining step, wherein a fluorescence signal value ratio of the at least two fluorophores is calculated to determine whether the at least one target is present in the sample.

2. The method for detecting multiple targets in the same sample of claim 1, wherein when the number of the at least two fluorophores is 2, the at least one target comprises a first target, a second target and a third target, the plurality of target primer pairs comprise a first primer pair, a second primer pair and a third primer pair, the plurality of target fluorescent probes comprise a first fluorescent probe, a second fluorescent probe and a third fluorescent probe, the at least two fluorophores comprise a first fluorophore and a second fluorophore, the first fluorophore and the second fluorophore are different, the first fluorescent probe comprises a first oligonucleotide bound to the first fluorophore, the second fluorescent probe comprises a second oligonucleotide bound to the second fluorophore, and the third fluorescent probe comprises a third oligonucleotide bound to the first fluorophore and the third oligonucleotide bound to the second fluorophore;
wherein the first primer pair and the first oligonucleotide respectively have sequence specificity with the first target, the second primer pair and the second oligonucleotide respectively have sequence specificity with the second target, and the third primer pair and the third oligonucleotide respectively have sequence specificity with the third target; and
wherein in the reaction step, the fluorescence signal values of the at least two fluorophores comprise a first fluorescence signal value and a second fluorescence signal value.

3. The method for detecting multiple targets in the same sample of claim 2, wherein a ratio of a content of the first fluorophore in the first fluorescent probe, a content of the second fluorophore in the second fluorescent probe and a total content of the first fluorophore and the second fluorophore in the third fluorescent probe is 1:1:1, and a ratio of the first fluorophore to the second fluorophore in the third fluorescent probe is 0.1:0.9 to 0.9:0.1.

4. The method for detecting multiple targets in the same sample of claim 3, wherein,
when the fluorescence signal value ratio is 1:0, only the first target is present in the sample;
when the fluorescence signal value ratio is 0:1, only the second target is present in the sample;
when the fluorescence signal value ratio is 0.1:0.9 to 0.9:0.1, only the third target is present in the sample;
when the fluorescence signal value ratio is 1.1:0.9 to 1.9:0.1, the first target and the third target are present in the sample;
when the fluorescence signal value ratio is 1:1, the first target and the second target are present in the sample;
when the fluorescence signal value ratio is 0.1:1.9 to 0.9:1.1, the second target and the third target are present in the sample; and
when the fluorescence signal value ratio is 1.1:1.9 to 1.9:1.1, the first target, the second target and the third target are present in the sample.

5. The method for detecting multiple targets in the same sample of claim 1, wherein the at least two fluorophores comprise at least two selected from a group consisting of FAM, FITC, Alexa 488, CF488, Atto 488, STAR 488, JOE, TET, Yakima Yellow, HEX, VIC, Atto 532, Alexa 532, Quasar 570, Cy3, TAMRA, ROX, Texas Red, Atto 590, Atto 594, Cy5, Atto 647, Alexa 647, Quasar 670 and Quasar 705.

6. The method for detecting multiple targets in the same sample of claim 2, wherein the first fluorescent probe, the second fluorescent probe and the third fluorescent probe respectively comprise a fluorescent quenching group, and the at least two fluorophores and the fluorescent quenching group are respectively bound to a 5′ end and a 3′ end of the first fluorescent probe, the second fluorescent probe and the third fluorescent probe.

7. The method for detecting multiple targets in the same sample of claim 1, wherein the sample comprises an internal control target as an internal control, the primer pair set comprises an internal control primer pair, the internal control primer pair has sequence specificity with the internal control target, the fluorescent probe set comprises an internal control fluorescent probe, the internal control fluorescent probe comprises an internal control oligonucleotide bound to the at least two fluorophores, and the internal control oligonucleotide has sequence specificity with the internal control target.

8. The method for detecting multiple targets in the same sample of claim 7, wherein Tx represents the at least one target, ic represents the internal control, Fm represents one of the at least two fluorophores, a content of each of the at least two fluorophores in the internal control fluorescent probe is α_{Fm}^{ic}, a content of each of the at least two fluorophores in each of the target fluorescent probes is β_{Fm}^{Tx}, and α_{Fm}^{ic}:β_{Fm}^{Tx} is 1:9, 2:8, 3:7, 4:6 or 5:5.

9. A method for quantifying concentration of multiple targets in the same sample, comprising:
performing a qualitative step, wherein whether the first target, the second target and/or the third target are present in the sample is determined by the method for detecting multiple targets in the same sample of claim 2; and
performing a quantitative step, wherein the first fluorescence signal value and the second fluorescence signal value are substituted into a calibration curve equation to calculate a concentration of the at least one target in the sample, the calibration curve equation is derived from a calibration curve set, and the calibration curve set comprises a calibration curve for detecting the first target using the first fluorophore, a calibration curve for detecting the first target using the second fluorophore, a calibration curve for detecting the second target using the first fluorophore, a calibration curve for detecting the second target using the second fluorophore, a calibration curve for detecting the third target using the first fluorophore, and a calibration curve for detecting the third target using the second fluorophore.

10. The method for quantifying concentration of multiple targets in the same sample of claim 9, wherein when the at least one target present in the sample is the first target, the second target or the third target, F1 represents the first fluorophore, F2 represents the second fluorophore, Tx represents the at least one target, and the calibration curve equation is expressed as equation (1):
$${S}_{F 1}^{Tx} + {S}_{F 2}^{Tx} = {ε}_{F 1}^{Tx} [ Tx ] + {ε}_{F 2}^{Tx} [ Tx ] + {C}_{F 1}^{Tx} + {C}_{F 2}^{Tx} ; \qquad ( 1 )$$
where and are respectively the first fluorescence signal value and the second fluorescence signal value of the at least one target, [Tx] is the concentration of the at least one target, ϵ_{F1}^{Tx} and ϵ_{F2}^{Tx} are respectively a slope of a calibration curve for detecting the at least one target using the first fluorophore and a slope of a calibration curve for detecting the at least one target using the second fluorophore, and C_{F1}^{Tx} and C_{F2}^{Tx} are respectively an intercept of the calibration curve for detecting the at least one target using the first fluorophore and an intercept of the calibration curve for detecting the at least one target the second fluorophore.

11. The method for quantifying concentration of multiple targets in the same sample of claim 9, wherein when the at least one target present in the sample are two of the first target, the second target and the third target, F1 represents the first fluorophore, F2 represents the second fluorophore, Tx represents one of the at least one target, Ty represents the other one of the at least one target, and the calibration curve equation is expressed as equation (2) and equation (3):
$${S}_{F 1}^{Tx} + {S}_{F 1}^{Ty} = {ε}_{F 1}^{Tx} [ Tx ] + {ε}_{F 1}^{Ty} [ Ty ] + {C}_{F 1}^{Tx} + {C}_{F 1}^{Tx} ; \qquad ( 2 )$$
$${S}_{F 2}^{Tx} + {S}_{F 2}^{Ty} = {ε}_{F 2}^{Tx} [ Tx ] + {ε}_{F 2}^{Ty} [ Ty ] + {C}_{F 2}^{Tx} + {C}_{F 2}^{Tx} ; \qquad ( 3 )$$
where S_{F1}^{Tx}, S_{F1}^{Ty}, and S_{F2}^{Ty} are respectively a first fluorescence signal value of the one of the at least one target, a first fluorescence signal value of the other one of the at least one target, a second fluorescence signal value of the one of the at least one target, and a second fluorescence signal value of the other one of the at least one target, [Tx] and [Ty ] are respectively a concentration of the one of the at least one target and a concentration of the other one of the at least one target, ϵ_{F1}^{Tx}, ϵ_{F1}^{Ty}, ϵ_{F2}^{Tx} and ϵ_{F2}^{Ty} are respectively a slope of a calibration curve for detecting the one of at least one target using the first fluorophore, a slope of a calibration curve for detecting the other one of at least one target using the first fluorophore, a slope of a calibration curve for detecting the one of at least one target using the second fluorophore, and a slope of a calibration curve for detecting the other one of at least one target using the second fluorophore, and C_{F1}^{Tx}, C_{F1}^{Ty}, C_{F2}^{Tx} and C_{F2}^{Ty} are respectively an intercept of the calibration curve for detecting the one of at least one target using the first fluorophore, an intercept of the calibration curve for detecting the other one of at least one target using the first fluorophore, an intercept of the calibration curve for detecting the one of at least one target using the second fluorophore, and an intercept of the calibration curve for detecting the other one of at least one target using the second fluorophore.

12. The method for quantifying concentration of multiple targets in the same sample of claim 9, wherein when the first target, the second target and the third target are present in the sample simultaneously, an auxiliary step is further performed before the quantitative step;
wherein in the auxiliary step, the target fluorescent probes in the reaction solution are adjusted such that the first fluorescent probe comprises the first oligonucleotide bound to the first fluorophore and the first oligonucleotide bound to the second fluorophore, or the second fluorescent probe comprises the second oligonucleotide bound to the first fluorophore and the second oligonucleotide bound to the second fluorophore, and the third fluorescent probe comprises the third oligonucleotide bound to the first fluorophore or the third oligonucleotide bound to the second fluorophore, and then the reaction solution and the sample are mixed and the real-time polymerase chain reaction is performed to obtain the other first fluorescent signal value of the first fluorophore and the other second fluorescent signal value of the second fluorophore.

13. The method for quantifying concentration of multiple targets in the same sample of claim 12, wherein in the quantitative step, the first fluorescent signal value, the second fluorescent signal value, the other first fluorescent signal value and the other second fluorescent signal value obtained in the qualitative step and the auxiliary step are substituted into the calibration curve equation to calculate a concentration of the first target, a concentration of the second target and a concentration of the third target in the sample;
wherein F1 represents the first fluorophore, F2 represents the second fluorophore, T1 represents the first target, T2 represents the second target, T3 represents the third target, and the calibration curve equation is expressed as equation (4) and equation (5):
$${S}_{F 1}^{T 1} + {S}_{F 1}^{T 2} + {S}_{F 1}^{T 3} = {ε}_{F 1}^{T 1} [ T 1 ] + {ε}_{F 1}^{T 2} [ T 2 ] + {ε}_{F 1}^{T 3} [ T 3 ] + {C}_{F 1}^{T 1} + {C}_{F 1}^{T 2} + {C}_{F 1}^{T 3} ; \qquad ( 4 )$$
$${S}_{F 2}^{T 1} + {S}_{F 2}^{T 2} + {S}_{F 2}^{T 3} = {ε}_{F 2}^{T 1} [ T 1 ] + {ε}_{F 2}^{T 2} [ T 2 ] + {ε}_{F 2}^{T 3} [ T 3 ] + {C}_{F 2}^{T 1} + {C}_{F 2}^{T 2} + {C}_{F 2}^{T 3} ; \qquad ( 5 )$$
where S_{F1}^{T1}, S_{F1}^{T2}, S_{F1}^{T3}, S_{F2}^{T1}, S_{F2}^{T2} and S_{F2}^{T3} are respectively the first fluorescence signal value of the first target, the first fluorescence signal value of the second target, the first fluorescence signal value of the third target, the second fluorescence signal value of the first target, the second fluorescence signal value of the second target, and the second fluorescence signal value of the third target, [T1], [T2] and [T3] are respectively the concentration of the first target, the concentration of the second target, and the concentration of the third target, ϵ_{F1}^{T1}, ϵ_{F1}^{T2}, ϵ_{F1}^{T3}, ϵ_{F2}^{T1}, ϵ_{F2}^{T2} and ϵ_{F2}^{T3} are respectively a slope of the calibration curve for detecting the first target using the first fluorophore, a slope of the calibration curve for detecting the second target using the first fluorophore, a slope of the calibration curve for detecting the third target using the first fluorophore, a slope of the calibration curve for detecting the first target using the second fluorophore, a slope of the calibration curve for detecting the second target using the second fluorophore, and a slope of the calibration curve for detecting the third target using the second fluorophore, and C_{F1}^{T1}, C_{F1}^{T2}, C_{F1}^{T3}, C_{F2}^{T1}, C_{F2}^{T2} and C_{F2}^{T3} are respectively an intercept of the calibration curve for detecting the first target using the first fluorophore, an intercept of the calibration curve for detecting the second target using the first fluorophore, an intercept of the calibration curve for detecting the third target using the first fluorophore, an intercept of the calibration curve for detecting the first target using the second fluorophore, an intercept of the calibration curve for detecting the second target using the second fluorophore, and an intercept of the calibration curve for detecting the third target using the second fluorophore.
